(19) United States
(12) Patent Application Publication
(10) Pub. No.: US 2021/0123869 A1
(43) Pub. Date: Apr. 29, 2021

(54) METHOD FOR MEASURING PH OF TEST WATER

(71) Applicant: MIURA CO., LTD., Matsuyama-shi, Ehime (JP)

(72) Inventors: Yusuke HAMADA, Matsuyama-shi (JP); Yuki ISHIHARA, Matsuyama-shi (JP)

(73) Assignee: MIURA CO., LTD., Matsuyama-shi, Ehime (JP)

(21) Appl. No.: 17/042,731

(22) PCT Filed: Jun. 28, 2019
(86) PCT No.: PCT/JP2019/025947

§ 371 Date: Sep. 28, 2020

(30) Foreign Application Priority Data
Jul. 3, 2018 (JP) 2018-126856

(51) Int. Cl. G01N 21/80; G01N 33/18

(52) U.S. Cl. CPC G01N 21/80; G01N 33/18

(57) ABSTRACT

A reagent composition prepared by dissolving, in a diol such as ethylene glycol, prescribed ratios of methyl red with an acid dissociation constant (pKa) of 5.1, phenol red with a pKa of 7.7 which is greater than that of methyl red, and bromocresol purple with a pKa of 6.3 which is between those of methyl red and phenol red is added to test water. With respect to the test water to which the reagent composition has been added, absorbances at three wavelengths of a wavelength selected from a range of from 410 to 430 nm, a wavelength selected from a range of from 515 to 535 nm, and a wavelength selected from a range of from 580 to 600 nm are measured, and the pH of the test water is determined based on the absorbances. In this way, it is possible to measure the pH of the test water in a range of from 4 to 9.

### TECHNICAL FIELD

The present invention relates to a method for measuring the pH of test water, and particularly to a method for measuring the pH of test water in a prescribed range. This application claims priority from Japanese Patent Application No. 2018-126856 filed in Japan on Jul. 3, 2018, the entire contents of which are hereby incorporated by reference.

### BACKGROUND ART

The pH (hydrogen ion exponent) of water for various purposes, such as water supplied to a boiler and circulating cooling water in a cooling tower, may be adjusted by adding a chemical agent. In this case, it is necessary to measure the pH of the water to which the chemical agent has been added, to confirm that the pH of the water is adjusted to be within a target range.

As a general method for measuring the pH of such water or a solution, Patent Literature 1 discusses a titration method and a measuring method using a glass electrode. However, in the titration method, as noted in Patent Literature 1, when the sample, i.e., the test water includes a large amount of metal component, a precipitate may be generated as titration proceeds. If a treatment for avoiding the influence of the precipitate is performed, issues of difficulty in detecting the end point of titration, operational complexity, and a need for a larger amount of the sample may arise. While the method employing a glass electrode provides a wide pH measurement range, it lacks a self-diagnostic function with respect to the measured value, and requires frequent checking and calibration to ensure the reliability of the measured value.

Thus, Patent Literature 1 discloses, as an alternative method capable of eliminating the disadvantages of the titration method and the method employing a glass electrode, a method for measuring the hydrogen ion concentration of the sample by adding a pH indicator to the test water and noting a change in absorbance associated with a color change of the test water. However, because the color-changing-range of a pH indicator is limited to a certain range, the width of pH that can be measured by the alternative method is narrow on the order of 1 to 2 at most.

### PRIOR ART LITERATURE

### Patent Literature

Patent Literature 1: JP-A-58-204343

### DISCLOSURE OF THE INVENTION

### Problems to be Solved by the Invention

The present invention makes it possible to measure the pH of test water in a relatively wide range using coloring reagents of which absorbance can be varied due to a pH variation.

### Solutions to the Problems

The present invention relates to a method for measuring the pH of test water in a prescribed range. The measuring method includes: a step 1 for adding, to the test water, a reagent composition including a first coloring reagent of which absorbance in an ultraviolet-visible region can be varied through acid dissociation in one stage due to a pH variation in the prescribed range, a second coloring reagent of which absorbance in the ultraviolet-visible region can be varied through acid dissociation in one stage due to a pH variation in the prescribed range and which has an acid dissociation constant (pKa) greater than that of the first coloring reagent, and at least one type of a third coloring reagent of which absorbance in the ultraviolet-visible region can be varied through acid dissociation in one stage due to a pH variation in the prescribed range and which has an acid dissociation constant (pKa) between those of the first coloring reagent and the second coloring reagent, each of the first coloring reagent, the second coloring reagent, and the third coloring reagent having an absorbance in the ultraviolet-visible region of greater than zero in the prescribed range; and a step 2 for measuring, with respect to the test water to which the reagent composition has been added, absorbance at an arbitrary wavelength in the ultraviolet-visible region; and a step 3 for determining the pH of the test water based on the absorbance measured in step 2.

An embodiment of the measuring method of the present invention may include: in step 2, measuring the absorbance at each of a plurality of mutually different wavelengths; and in step 3, determining the pH of the test water in accordance with a correlation analysis result obtained in advance using, as variables, the absorbance at each of the plurality of wavelengths and pH of the test water.

Another embodiment of the measuring method of the present invention may include: in step 2, measuring the absorbance at each of a plurality of mutually different, at least three, wavelengths; and in step 3, determining the pH of the test water in accordance with a correlation analysis result obtained in advance using, as variables, absorbance ratios in which the denominator is the absorbance with respect to a wavelength among the plurality of wavelengths at which the absorbance is least likely to change due to a variation in the pH of the test water, and in which the numerator each separately is the absorbance with respect to each of the other wavelengths, and pH of the test water.

A modification of the measuring method according to the other embodiment may include: in step 3, provisionally determining, separately for each of the absorbance ratios, a pH of the test water in accordance with a correlation analysis result obtained in advance using, as variables, each one of the absorbance ratios and pH of the test water; and cancelling step 3 if a difference between the pH of the test water provisionally determined based on one of the absorbance ratios and the pH of the test water provisionally determined based on another one of the absorbance ratios is greater than a prescribed value.

A modification of the measuring method of the present invention may further include a step 4 in which step 1 to step 3 are repeated at least once to set a function (y=Fx) of a pH (y) of the test water determined in each step 3 and a cumulative added amount (x) of the reagent composition with respect to the test water at the time of the determination as variables, and in which the pH (y) when the added amount (x) is zero in the function (y=Fx) is finally determined as the pH of the test water.

This modification may include determining a buffer capacity of the test water based on the function (y=Fx). Preferably, the modification may include using, as the reagent composition added to the test water in step 1, one that includes an amino acid if the buffer capacity determined with respect to the test water is greater than a prescribed value.

In an example of the measuring method of the present invention, the prescribed range of pH may be from 4 to 9. This example may include: in step 1, using the reagent composition that includes a first coloring reagent selected from those with an acid dissociation constant (pKa) in a range of from 4.1 to 6.0, a second coloring reagent selected from those with an acid dissociation constant (pKa) in a range of from 6.5 to 8.5, and one type of a third coloring reagent selected from those with an acid dissociation constant (pKa) in a range of from 5.5 to 7.5; and, in step 2, measuring absorbance at three wavelengths of a wavelength selected from a range of from 410 to 430 nm, a wavelength selected from a range of from 515 to 535 nm, and a wavelength selected from a range of from 580 to 600 nm.

In another example of the measuring method of the present invention, the prescribed range of pH may be from 4 to 12. This example may include: in step 1, using the reagent composition that includes a first coloring reagent selected from those with an acid dissociation constant (pKa) in a range from 4.1 to 6.0, a second coloring reagent selected from those with an acid dissociation constant (pKa) in a range of from 8.5 to 11.5, and a total of two types of a third coloring reagent including a first type of coloring reagent selected from those with an acid dissociation constant (pKa) in a range of from 5.5 to 7.5, and a second type of coloring reagent selected from those with an acid dissociation constant (pKa) in a range of from 7.0 to 9.5 and having an acid dissociation constant (pKa) greater than that of the first type of coloring reagent; and in step 2, measuring absorbance at four wavelengths of a wavelength selected from a range of from 410 to 430 nm, a wavelength selected from a range of from 450 to 470 nm, a wavelength selected from a range of from 515 to 535 nm, and a wavelength selected from a range of from 580 to 600 nm.

### Effects of the Invention

In a method for measuring the pH of test water according to the present invention, a reagent composition including at least three types of coloring reagents of which absorbance can be varied through acid dissociation in one stage due to a pH variation and which have mutually different acid dissociation constants (pKa) is added to the test water, the absorbance at an arbitrary wavelength in the ultraviolet-visible region is measured, and then the pH of the test water is determined based on the absorbance. Accordingly, it is possible to measure the pH of the test water in a relatively wide range.

### BRIEF DESCRIPTION OF THE DRAWINGS

FIG. 1 shows absorption spectra of methyl red.

FIG. 2 shows absorption spectra of phenol red.

FIG. 3 shows absorption spectra of bromocresol purple.

FIG. 4 is a graph illustrating the color-changing pH regions of coloring reagents included in a reagent composition according to a concrete example of a first embodiment.

FIG. 5 shows absorption spectra of bromophenol blue.

FIG. 6 shows absorption spectra of alizarin yellow.

FIG. 7 is a graph illustrating the color-changing pH regions of coloring reagents included in a reagent composition according to a concrete example of a second embodiment.

FIG. 8 is a schematic graph illustrating a relationship between a change in the amount of the reagent composition added to the test water and the pH of the test water when step 1 to step 3 of a measuring method according to the present invention are repeated.

FIG. 9 is a pH determination graph created in an Example.

### DESCRIPTION OF PREFERRED EMBODIMENTS

A pH measuring method of the present invention is a method for measuring the pH of test water collected from water for various purposes, such as water supplied to a boiler and circulating cooling water for a cooling tower, or from various aqueous solutions, in a certain limited range (which may be referred to as a “prescribed range”). The pH measuring method includes the following steps 1 to 3.

### Step 1:

In this step, a prescribed reagent composition is added to the test water. Preferably, the test water to which the reagent composition has been added is stirred, as appropriate, so that the added reagent composition is dispersed evenly.

The reagent composition used herein includes a first coloring reagent, a second coloring reagent, and a third coloring reagent. Each of the coloring reagents used herein has a different degree of acid dissociation and shows a change in abundance ratio between the base type (HIn) that has not been acid-dissociated and the acid type (In−) that has been acid-dissociated, depending on the pH of the environment in which the coloring agent exists, resulting in a change in absorbance in an ultraviolet-visible region with respect to the existing environment. When this type of coloring reagent is added to the test water, if the pH of the test water is in a pH range in which acid dissociation of the coloring reagent can occur, it is possible, by measuring the absorbance at an arbitrary wavelength of the ultraviolet-visible region with respect to the test water, to determine the abundance ratio of the acid type (In−) to the base type (HIn) of the coloring reagent in the test water. Thus, it is possible to calculate the pH of the test water from the abundance ratio and the acid dissociation constant (pKa) of the coloring reagent, based on the following Henderson-Hasselbalch's equation, where pKa is a value at 25° C.:

$$pH = p Ka + {log}_{10} \frac{[ {In}^{-} ]}{[ HIn ]} \qquad [ Equation 1 ]$$

Each of the coloring reagents used in the reagent composition is one of which absorbance in the ultraviolet-visible region can be varied through acid dissociation in one stage due to a pH variation in the prescribed range, and in which the absorbance in the ultraviolet-visible region in the prescribed range is more than zero such that absorption of the ultraviolet-visible region is not eliminated in the prescribed range.

As is obvious in view of the Henderson-Hasselbalch's equation, the pH region in which acid dissociation can occur differs depending on the pKa of the coloring reagent. Accordingly, in order to ensure a certain width as a prescribed range of measurable pH, the first coloring reagent, the second coloring reagent, and the third coloring reagent used in the reagent composition have mutually different pKas. That is, as the second coloring reagent, one with a pKa greater than that of the first coloring reagent is selected. As the third coloring reagent, one with a pKa between those of the first coloring reagent and the second coloring reagent is selected. The third coloring reagent may consist of one type of coloring reagent, or may consist of two or more types of coloring reagents. When one type of coloring reagent is used as the third coloring reagent, the pKa of the coloring reagent is preferably an approximately central value between the pKa of the first coloring reagent and the pKa of the second coloring reagent. When two or more types of coloring reagents are used as the third coloring reagent, coloring reagents having mutually different pKas are selected. In this case, the respective coloring reagents in the third coloring reagent preferably have pKas of which the values are at approximately equal intervals between the pKa of the first coloring reagent and the pKa of the second coloring reagent.

Embodiments of the reagent composition include a first embodiment and a second embodiment described below.

The absorption spectrum of each of the coloring reagents selected in concrete examples of the respective embodiments is measured with respect to a solution obtained by diluting, 150-fold with a dilution water (for example, distilled water), a reagent obtained by adjusting the concentration of the coloring reagent to 1.00 g/kg (hereafter, the concentration of a coloring reagent in a solution prepared as described above may be referred to as a “unit concentration of coloring reagent”). For absorption spectrum measurement, a spectrophotometer by Hitachi High-Tech Science Corporation (Type: U-2910) was used, where the measured wavelength range was set to 350 nm to 800 nm using a cell with an optical path length of 10 mm. With respect to each coloring reagent, the base type means a state before acid dissociation, and the acid type means a state after acid dissociation. The strong acid type of phenol red means a state after a second stage of acid dissociation, as will be described below.

The following first embodiment, second embodiment, and their concrete examples do not limit the reagent composition used in the measuring methods of the present invention.

### First Embodiment

The present embodiment makes it possible to measure the pH of the test water in a range of generally from 4 to 9 (this range includes the entire carbonate buffer pH region), and includes the following coloring reagents.
First coloring reagent

This is selected from coloring reagents with pKa in a range of from 4.1 to 6.0. For example, this may be selected from the group consisting of methyl red (pKa: 5.1), bromophenol blue (pKa: 4.2), and bromocresol green (pKa: 4.7).
Second coloring reagent

This is selected from coloring reagents with pKa in a range of from 6.5 to 8.5. For example, this may be selected from the group consisting of phenol red (pKa: 1.2 and 7.7), neutral red (pKa: 6.7 and 7.4), and cresol red (pKa: 1.0 and 8.0).
Third coloring reagent

This is selected from coloring reagents with pKa in a range of from 5.5 to 7.5. For example, this may be selected from the group consisting of bromocresol purple (pKa: 6.3) and bromthymol blue (pKa: 7.1).

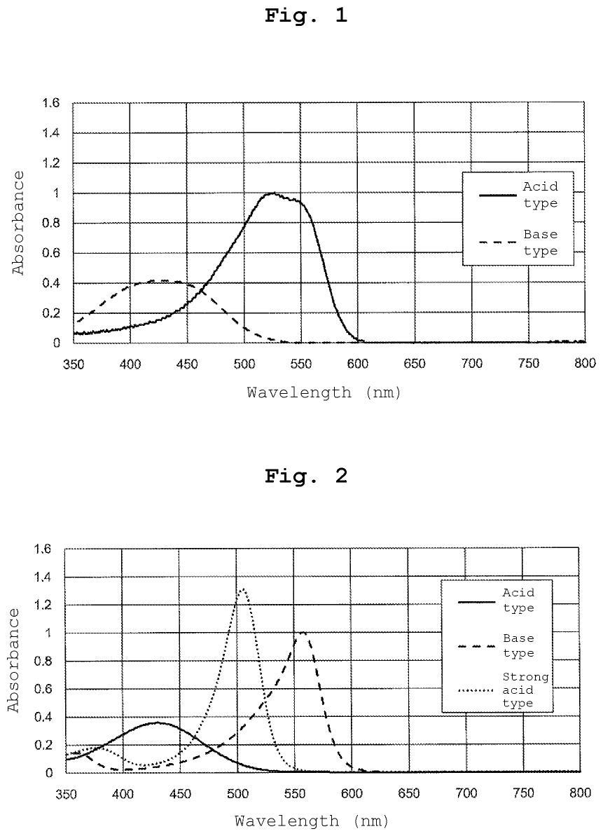

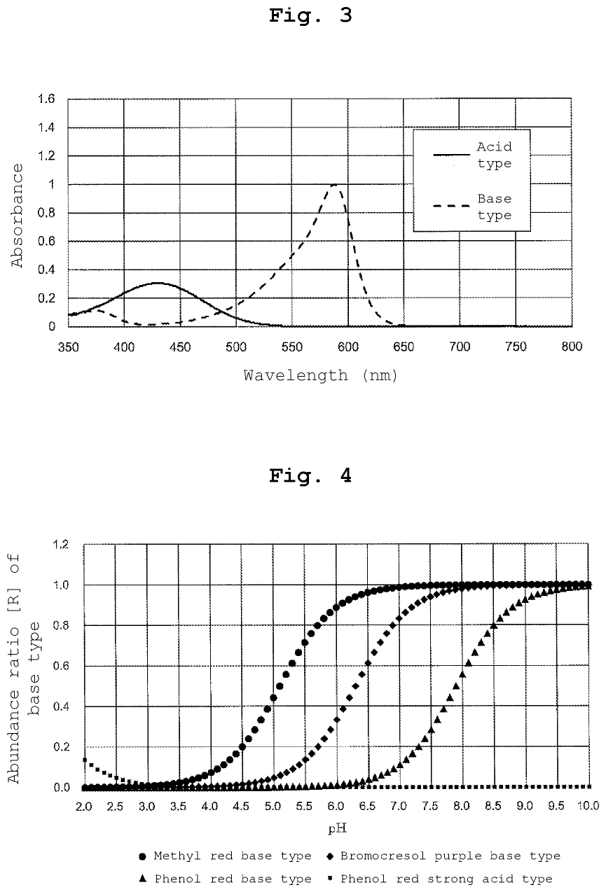

In a concrete example of the present embodiment, the reagent composition includes the following coloring reagents:
First coloring reagent
   Methyl red
   pKa: 5.1
   Absorption spectrum: FIG. 1
Second coloring reagent
   Phenol red
   pKa: 1.2 and 7.7
   Absorption spectrum: FIG. 2
Third coloring reagent
   Bromocresol purple
   pKa: 6.3
   Absorption spectrum: FIG. 3

With respect to each of the coloring reagents included in the reagent composition of the concrete example, color-changing pH regions determined from pKa on the basis of the Henderson-Hasselbalch's equation are shown in FIG. 4. According to FIG. 4, methyl red of the first coloring reagent can change color in a pH range of generally from 4 to 6; phenol red of the second coloring reagent can change color in a pH range of generally from 7 to 9; and bromocresol purple of the third coloring reagent can change color in a pH range of generally from 5.5 to 7. Thus, with the reagent composition of the concrete example, it is possible to measure the pH of the test water in a prescribed range of generally from 4 to 9.

Phenol red can be acid-dissociated in two stages depending on the pH of the existing environment, and therefore has two pKas. However, one pKa (7.7) is greater than the pKa (5.1) of methyl red used as the first coloring reagent and is greater than the pKa (6.3) of bromocresol purple used as the third coloring reagent, and the acid dissociation in the prescribed pH range of from 4 to 9 occurs in one stage. Accordingly, phenol red can be used as the second coloring reagent.

### Second Embodiment

The present embodiment makes it possible to measure the pH of the test water in a range of generally from 4 to 12 (this range also includes the entire carbonate buffer pH region), and includes the following coloring reagents:
First coloring reagent

This is selected from coloring reagents with pKa in a range of from 4.1 to 6.0. For example, this may be selected from the group consisting of methyl red (pKa: 5.1), bromophenol blue (pKa: 4.2), and bromocresol green (pKa: 4.7).
Second coloring reagent

This is selected from coloring reagents with pKa in a range of from 8.5 to 11.5. For example, this may be selected from the group consisting of alizarin yellow (pKa: 11.06) and thymol blue (pKa: 1.7 and 8.9).
Third coloring reagent

This includes the two types of a coloring reagent A selected from coloring reagents with pKa in a range of from 5.5 to 7.5, and a coloring reagent B selected from coloring reagents with pKa in a range of from 7.0 to 9.5. Note, however, that the coloring reagent B that is selected has a pKa greater than that of the coloring reagent A. The coloring reagent A may be selected from the group consisting of bromocresol purple (pKa: 6.3) and bromthymol blue (pKa: 7.1), for example. The coloring reagent B may be selected from the group consisting of phenol red (pKa: 1.2 and 7.7), neutral red (pKa: 6.7 and 7.4), and cresol red (pKa: 1.0 and 8.0), for example.

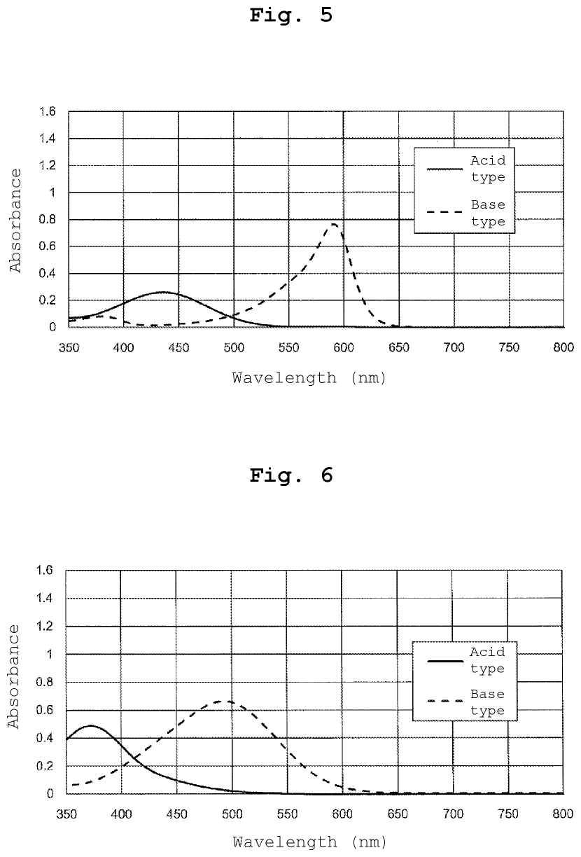

In a concrete example of the present embodiment, the reagent composition includes the following coloring reagents:
First coloring reagent
   Bromophenol blue
   pKa: 4.2
   Absorption spectrum: FIG. 5
Second coloring reagent
   Alizarin yellow
   pKa: 11.06
   Absorption spectrum: FIG. 6
Third coloring reagents: the following two types of a coloring reagent A and a coloring reagent B
   Coloring reagent A
Bromocresol purple
pKa: 6.3
Absorption spectrum: FIG. 3
   Coloring reagent B
Phenol red
pKa: 1.2 and 7.7
Absorption spectrum: FIG. 2

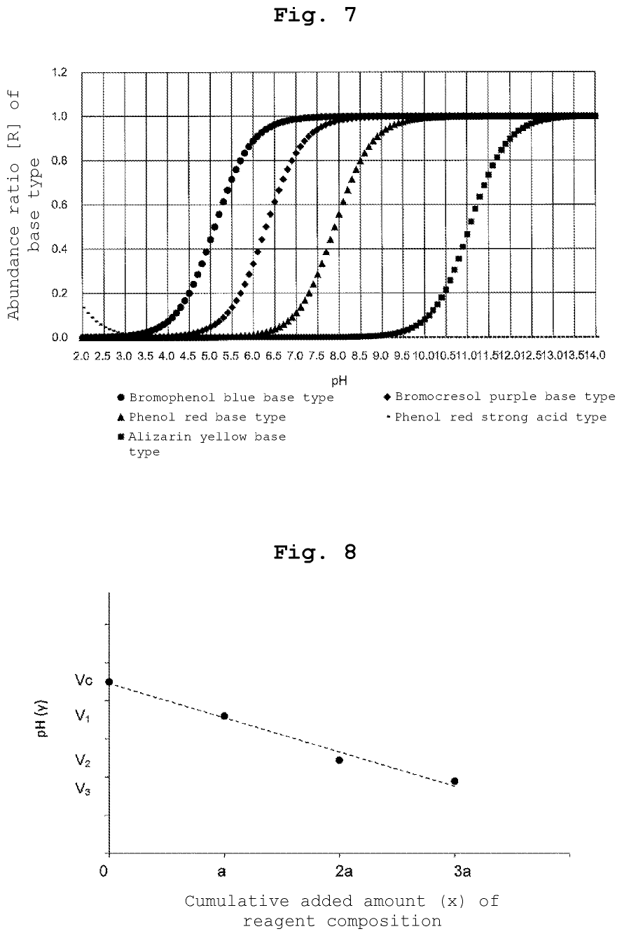

With respect to each of the coloring reagents included in the reagent composition of the concrete example, color-changing pH regions determined from pKa on the basis of the Henderson-Hasselbalch's equation are shown in FIG. 7. According to FIG. 7, bromophenol blue of the first coloring reagent can change color in a pH range of generally from 3 to 5; alizarin yellow of the second coloring reagent can change color in a pH range of generally from 9 to 12; and, of the third coloring reagents, bromocresol purple of the coloring reagent A can change color in a pH range of generally from 5 to 7, and phenol red of the coloring reagent B can change color in a pH range of generally from 7 to 9. Accordingly, with the reagent composition of the concrete example, it is possible to measure the pH of the test water in a prescribed range of generally from 4 to 12.

While phenol red has two pKas as noted above, one pKa (7.7) is greater than the pKa (3.85) of the bromophenol blue used as the first coloring reagent and is smaller than the pKa (11.06) of the alizarin yellow used as the second coloring reagent, and the acid dissociation in the prescribed pH range of from 4 to 12 occurs in one stage. Thus, it is possible to use phenol red as one of the third coloring reagents. With respect to the other coloring reagents having two pKas (for example, thymol blue, neutral red, and cresol red), any of the coloring reagents may be used as a required coloring reagent if one pKa thereof satisfies the requirements as the first coloring reagent, the second coloring reagent, or the third coloring reagent.

In the reagent composition, it is basically preferable to set the blending ratios of the respective coloring reagents to be equimolar. However, the amount of a coloring reagent with a pKa closer to a pH for which higher resolution (determination accuracy) is desirable may be set higher.

The reagent composition is usually obtained by dissolving the required coloring reagents in a solvent. Various types of solvent may be used, as long as the solvent when added to the test water is not likely to affect the absorbance of the coloring reagents. For example, it is possible to use purified water such as distilled water and pure water; and diols such as ethylene glycol, propylene glycol, and propanediol. The reagent composition may include a surfactant in order to suppress attachment of dirt to a cell for absorbance measurement used in the next step 2. Various types of surfactant may be used, such as cationic, anionic, and non-ionic surfactants. Among others, a non-ionic surfactant is preferable.

In this step, the amount of the reagent composition added to the test water is set to a prescribed amount determined in advance. The prescribed amount is with reference to a total amount of the respective coloring reagents, and may be hereafter referred to as a “reference added amount”.

### Step 2:

In this step, with respect to the test water to which the reagent composition has been added in step 1, absorbance at a specific wavelength (which may be hereafter referred to as “the specific wavelength”) selected as desired from the ultraviolet-visible region is measured. Here, required absorbance is measured by irradiating the test water with the specific wavelength of light and receiving the light that has passed through the test water. In this case, a readily available light source may be used for measuring the absorbance. For example, it is possible to use a light-emitting diode (LED) that emits the specific wavelength of light from a group of various LEDs having different colors of emitted light. The absorbance measurement may also involve measuring an absorption spectrum by irradiating the test water with light of wavelengths of the ultraviolet-visible light region, typically 100 nm to 800 nm, using a spectrophotometer, and then determining the absorbance at the specific wavelength from the absorption spectrum.

The specific wavelength is not particularly limited. However, it is preferable to adopt a wavelength that facilitates observation of an absorbance change in consideration of: stable absorption even when there is a little shift in wavelength, while allowing for strong absorption by the object being measured; and the fact that the pH measurement range is prone to become narrower if there is too much change in the absorbance of the object being measured, while the accuracy of pH measurement tends to decrease if the change is too little.

In this step, absorbance at a single specific wavelength may be measured, or absorbance at a plurality of mutually different specific wavelengths may be measured.

### Step 3:

In this step, the pH of the test water is determined on the basis of the absorbance at the specific wavelength measured in step 2.

The absorbance at the specific wavelength with respect to the test water to which the reagent composition has been added in step 1 appears, theoretically, as a sum of the absorbance at the specific wavelength with respect to each of the coloring reagents included in the reagent composition added to the test water step 1. That is, the absorbance at the specific wavelength with respect to the test water to which the reagent composition has been added is an integration, for each concentration, of the absorbance at the specific wavelength for each of the base type and acid type of each of the coloring reagents included in the reagent composition. Accordingly, by using the reagent composition that includes the three types of coloring reagents of the first coloring reagent, the second coloring reagent, and the third coloring reagent consisting of one type of coloring reagent, and in which the blending ratios of the coloring reagents are known, it is possible, theoretically, to calculate the absorbance at the specific wavelength with respect to the test water to which the reagent composition has been added according to the following relational expression, in which the meaning of each symbol is as noted in Tables 1 and 2:

A_{λ}=D{β_{R} [symbol] _{Aλ}·(1−R1)+β_{R} [symbol] _{Aλ}·R1+β_{R} [symbol] _{ABλ}·(1−R3)+β_{R} [symbol] _{Bλ}·R3+β_{R} [symbol] _{Aλ}·(1−R2)+β_{R} [symbol] _{Bλ}·R2} [Equation 2]

TABLE 1

| Symbol | Meaning |
|---|---|
| β_{R1Aλ} | Absorbance of acid type of first coloring reagent at |
| | specific wavelength λ |
| β_{R1Bλ} | Absorbance of base type of first coloring reagent at |
| | specific wavelength λ |
| β_{R2Aλ} | Absorbance of acid type of second coloring reagent at |
| | specific wavelength λ |
| β_{R2Bλ} | Absorbance of base type of second coloring reagent at |
| | specific wavelength λ |
| β_{R3Aλ} | Absorbance of acid type of third coloring reagent at |
| | specific wavelength λ |
| β_{R3Bλ} | Absorbance of base type of third coloring reagent at |
| | specific wavelength λ |

Each absorbance in Table 1 is defined by a relationship (absorbance×blending ratio) between the absorbance at the specific wavelength with respect to a solution of a corresponding coloring reagent adjusted to the unit concentration of coloring reagent, and the blending ratio of the corresponding coloring reagent in the reagent composition.

TABLE 2

| Symbol | Unit | Range | Meaning |
|---|---|---|---|
| A_{λ} | Abs | 0 to 2 | Absorbance measurement result for |
| | | | specific wavelength λ |
| R1 | [—] | 0 to 1 | Abundance ratio of base type of first |
| | | | coloring reagent |
| R2 | [—] | 0 to 1 | Abundance ratio of base type of second |
| | | | coloring reagent |
| R3 | [—] | 0 to 1 | Abundance ratio of base type of third |
| | | | coloring reagent |
| D | | 0 to 2 | Deviation in amount of reagent |
| | | | composition added to test water (where |
| | | | reference added amount is 1) |

According to the Henderson-Hasselbalch's equation, the abundance ratio of the base type of each coloring reagent varies depending on the pH of the test water. Accordingly, it is possible to predict, on the basis of the relational expression for each pH of the test water, the absorbance at the specific wavelength in the test water when a reference injection amount of the reagent composition with known blending ratios of the coloring reagents has been injected into the test water. Thus, by predicting beforehand the absorbance at the specific wavelength for each pH of the test water in accordance with the reagent composition used in step 1, it is possible to determine the pH of the test water by comparing the result of the prediction with the absorbance at the specific wavelength actually measured in step 2.

When a plurality of mutually different specific wavelengths, such as two to five absorbances, is measured in step 2, a correlation between the pH of the test water to which the reagent composition has been added and the absorbance at each specific wavelength may be analyzed beforehand in light of the above relational expression and the Henderson-Hasselbalch's equation. In this way, it is possible to determine the pH of the test water more accurately in this step. For example, consider a case of using the reagent composition according to the first embodiment, which includes the three types of coloring reagents of the first coloring reagent, the second coloring reagent, and the third coloring reagent consisting of one type of coloring reagent, and of which the blending ratio of each coloring reagent is known. In this case, in light of the relational expression, three relational expressions (1), (2), and (3) indicated below hold between: absorbances at three specific wavelengths with respect to the test water to which the reagent composition has been added, i.e., absorbances at the three specific wavelengths λ1, λ2, and λ3 (where λ1<λ2<λ3); and the abundance ratios of the base type and acid type of each coloring reagent in the test water. The meaning of each symbol in the relational expressions (1), (2), and (3) is as noted in Tables 3 and 4.

[Equation 3]

A_{λ} [symbol] =D{β_{R} [symbol] _{Aλ} [symbol] ·(1−R1)+β_{R} [symbol] _{Bλ} [symbol] ·R1+β_{R} [symbol] _{Aλ} [symbol] ·(1−R3)+β_{R} [symbol] _{Bλ} [symbol] ·R3+β_{R} [symbol] _{Aλ} [symbol] ·(1−R2)+β_{R} [symbol] _{Bλ} [symbol] ·R2} (1)

A_{λ} [symbol] =D{β_{R} [symbol] _{Aλ} [symbol] ·(1−R1)+β_{R} [symbol] _{Bλ} [symbol] ·R1+β_{R} [symbol] _{Aλ} [symbol] ·(1−R3)+β_{R} [symbol] _{Bλ} [symbol] ·R3+β_{R} [symbol] _{Aλ} [symbol] ·(1−R2)+β_{R} [symbol] _{Bλ} [symbol] ·R2} (2)

A_{λ} [symbol] =D{β_{R} [symbol] _{Aλ} [symbol] ·(1−R1)+β_{R} [symbol] _{Bλ} [symbol] ·R1+β_{R} [symbol] _{Aλ} [symbol] ·(1−R3)+β_{R} [symbol] _{Bλ} [symbol] ·R3+β_{R} [symbol] _{Aλ} [symbol] ·(1−R2)+β_{R} [symbol] _{Bλ} [symbol] ·R2} (3)

TABLE 3

| Symbol | Meaning |
|---|---|
| β_{R1Aλ1}, β_{R1Aλ}2, and β_{R1Aλ3} | Absorbance of acid type of first |
| | coloring reagent at specific wavelengths |
| | λ1, λ2, and λ3, respectively |
| β_{R1Bλ1}, β_{R1Bλ2}, and β_{R1Bλ3} | Absorbance of base type of first |
| | coloring reagent at specific wavelengths |
| | λ1, λ2, and λ3, respectively |
| β_{R2Aλ1}, β_{R2Aλ2}, and β_{R2Aλ3} | Absorbance of acid type of second |
| | coloring reagent at specific wavelengths |
| | λ1, λ2, and λ3, respectively |
| β_{R2Bλ1}, β_{R2Bλ2}, and β_{R2Bλ3} | Absorbance of base type of second |
| | coloring reagent at specific wavelengths |
| | λ1, λ2, and λ3, respectively |
| β_{R3Aλ1}, β_{R3Aλ2}, and β_{R3Aλ3} | Absorbance of acid type of third |
| | coloring reagent at specific wavelengths |
| | λ1, λ2, and λ3, respectively |
| β_{R3Bλ1}, β_{R3Bλ2}, and β_{R3Bλ3} | Absorbance of base type of third |
| | coloring reagent at specific wavelengths |
| | λ1, λ2, and λ3, respectively |

Each absorbance in Table 3 is defined by a relationship (absorbance×blending ratio) between the absorbance at the corresponding specific wavelength with respect to a solution of the corresponding coloring reagent adjusted to the unit concentration of coloring reagent, and the blending ratio of the corresponding coloring reagent in the reagent composition.

TABLE 4

| Symbol | Unit | Range | Meaning |
|---|---|---|---|
| A_{λ1} | Abs | 0 to 2 | Result of absorbance measurement at |
| | | | specific wavelength λ1 |
| A_{λ2} | Abs | 0 to 2 | Result of absorbance measurement at |
| | | | specific wavelength λ2 |
| A_{λ3} | Abs | 0 to 2 | Result of absorbance measurement at |
| | | | specific wavelength λ3 |
| R1 | [—] | 0 to 1 | Abundance ratio of base type of first |
| | | | coloring reagent |
| R2 | [—] | 0 to 1 | Abundance ratio of base type of second |
| | | | coloring reagent |
| R3 | [—] | 0 to 1 | Abundance ratio of base type of third |
| | | | coloring reagent |
| D | | 0 to 2 | Deviation in amount of reagent |
| | | | composition added to test water (where |
| | | | reference added amount is 1) |

In this example, in light of the expressions (1), (2), and (3) as well as the Henderson-Hasselbalch's equation, correlations between the three specific wavelengths λ1, λ2, and λ3 and pH of the test water to which the reagent composition has been added may be analyzed in advance. In this way, it is possible to determine, in accordance with the analysis result, the pH of the test water on the basis of the results of absorbance measurement performed in step 2 at the three wavelengths λ1, λ2, and λ3.

In particular, when absorbances at a plurality of three or more specific wavelengths are measured, as in the above example, absorbance ratios in which the denominator is the absorbance at one specific wavelength and in which the numerator each separately is the absorbance with respect to each of the other specific wavelengths may be determined, and then it is possible to determine the pH of the test water in accordance with a correlation analysis result obtained in advance using, as variables, the absorbance ratios and pH of the test water. In this case, even if the amount of the reagent composition added to the test water in step 1 is varied from the reference added amount, it is possible to obtain a highly reliable determination result with respect to the pH of the test water in this step.

For example, when absorbances at the three specific wavelengths λ1, λ2, and λ3 are measured, as in the above example, the pH of the test water is determined in accordance with a correlation analysis result obtained in advance using, as variables: absorbance ratios in which the denominator is the absorbance at a specific wavelength (λ1, provisionally) among the specific wavelengths λ1, λ2, and λ3 at which the absorbance is least likely to change due to a variation in the pH of the test water, and in which the numerator each separately is the absorbance at each of the other specific wavelengths (λ2 and λ3, provisionally), namely, Aλ2/Aλ 1 (an absorbance ratio A) and Aλ3/Aλ1 (an absorbance ratio B); and pH of the test water.

When the pH of the test water is determined in accordance with the correlation analysis result in which the above-described absorbance ratios are adopted, the reliability of the determination result can be further increased. Here, pHs of the test water are provisionally determined from the results of absorbance measurement in step 2, in accordance with a correlation analysis result obtained in advance using, as variables, each of the absorbance ratios and pH of the test water. Then, the pHs of the test water provisionally determined based on the absorbance ratios are compared. If the difference between the pH of the test water provisionally determined based on one of the absorbance ratios and the pH of the test water provisionally determined based on another absorbance ratio is greater than a prescribed value, step 3 is cancelled because there may have been a formulation problem in the reagent composition added to the test water in step 1, the reagent composition may be degraded or deteriorated, or there may be some kind of abnormality in color development of the test water due to the reagent composition. For example, in the above-described example, a pH of the test water is provisionally determined from the result of absorbance measurement in step 2 in accordance with the correlation analysis result obtained in advance using, as variables, the absorbance ratio A and pH of the test water, and a pH of the test water is provisionally determined from the result of absorbance measurement in step 2 in accordance with a correlation analysis result obtained in advance using, as variables, the absorbance ratio B and pH of the test water. If the difference between the pH of the test water provisionally determined based on the absorbance ratio A and the pH of the test water provisionally determined based on the absorbance ratio B is greater than a prescribed value (for example, 0.5), step 3 is cancelled. The prescribed value of the pH difference may be set as desired, in accordance with the expected measurement accuracy.

The reagent composition added to the test water in step 1 may include four or more types of coloring reagents, and absorbance may be measured at a plurality of specific wavelengths in step 2. In this case, as in the above example, the correlation between absorbances at the plurality of specific wavelengths and pH of the test water to which the reagent composition has been added may be analyzed in advance, in light of the plurality of relational expressions relating to the absorbance at each specific wavelength and the Henderson-Hasselbalch's equation. In this way, it is possible to determine the pH of the test water in accordance with the analysis result on the basis of the results of absorbance measurement at each wavelength in step 2.

In this case, it is also possible to determine the pH of the test water using absorbance ratios, as in the above example. Further, when the need for cancellation of step 3 is determined by utilizing absorbance ratios, three or more absorbance ratios may be obtained. Accordingly, for example, two absorbance ratios are selected from the absorbance ratios, as desired, and step 3 is cancelled if the difference between the pHs of the test water provisionally determined from the respective absorbance ratios is greater than the prescribed value.

The measuring method of the present invention may further include the following step 4.

### Step 4:

According to the measuring method of the present invention, the reagent composition is added to the test water. Thus, the method is not capable of measuring the pH of the test water per se, but rather measures the pH of the test water including the reagent composition that has been added. Because the coloring reagents included in the reagent composition develop color due to acid dissociation, the coloring reagents act to vary the pH of the test water in a decreasing direction due to protons released into the test water. As a result, the inherent pH value of the test water may be varied. The degree of influence of the reagent composition on the pH of the test water varies depending on the buffer capacity of the test water. That is, when the buffer capacity is high (such as, typically, when a buffer component such as carbonate is included), the test water is less likely to show a pH variation due to the influence of the reagent composition; however, when the buffer capacity is low, the pH is more likely to vary due to the influence of the reagent composition. Accordingly, in the measuring method of the present invention, it is preferable to correct the measurement result so as to remove a pH variation due to the influence of the reagent composition.

During the correction of the measurement result, a series of operations from step 1 to step 3 is repeated at least once (that is, the series of operations from step 1 to step 3 is repeatedly performed twice or more), and the pH of the test water is determined in step 3 of each repeated operation. The reagent composition added in each step 1 acts to lower the pH of the test water, as noted above. Accordingly, the pH of the test water determined in step 3 of each repeated operation decreases in a stepwise manner as the reagent composition is added in a stepwise manner. For example, as schematically illustrated in FIG. 8, the pH of the test water, when the added amount of the reagent composition is set to a in step 1, shows a value V1 determined in the initial step 3, a value V2 determined in the second step 3 which is lower than the value V1, and a value V3 determined in the third step 3 which is even lower than the value V2.

Accordingly, a function (y=Fx) of a pH (y) of the test water determined in step 3 of each repeated operation and a cumulative added amount (x) of the reagent composition with respect to the test water at the time of the determination as variables is set, and the pH (y) when the added amount (x) is zero in the function (y=Fx) is determined finally as the pH of the test water. For example, when the function (y=Fx) is linear as indicated by a dotted line in FIG. 8, Vc which is the pH value when the added amount (x) is zero is determined as the pH value of the test water per se.

The buffer capacity of the test water can be evaluated in the light of a status of change in the pH of the test water determined in step 3 of each repeated operation during the correction operation. The status of change can be determined quantitatively in light of the function (y=Fx). When the buffer capacity of the test water is determined to be small, the pH of the test water will exhibit relatively pronounced variations in each step 3, making correction by the function (y=Fx) easy. However, when the buffer capacity of the test water is determined to be large, the pH of the test water will exhibit obscure variations in each step 3, possibly making appropriate correction by the function (y=Fx) difficult.

Thus, when it is determined that the buffer capacity of the test water is large, particularly when the buffer capacity determined in light of the function (y=Fx) is greater than a prescribed value which is set as desired, it is preferable to use, as the reagent composition added to the test water in step 1, one that includes an amino acid. An amino acid helps to increase the buffer capacity of the coloring reagents in the reagent composition, making it possible to facilitate a change in the pH of the test water to which the reagent composition has been added.

Specifically, when the pH of the test water is on the acid side (low pH), the amino acid turns into —NH3+ by having a proton (hydrogen ion) coordinated to an amino group (—NH2) thereof, thereby tending to increase the pH of the test water to which the reagent composition has been added toward neutral. On the other hand, when the pH of the test water is on the alkaline side (high pH), the amino acid, due to a proton (hydrogen ion) released from a carboxyl group (—COOH), tends to decrease the pH of the test water to which the reagent composition has been added toward neutral. For example, when the test water has a low pH due to a carbonate (H2CO3) content, some of hydrogen ions generated due to carbonate dissociation are coordinated to the amino group of the amino acid, so that, as the reagent composition is added, the pH of the test water increases and tends to change toward neutral. When the test water has a high pH due to an ammonia (NH3) content, some of hydroxyl ions (OH—) generated by ionization of ammonia in the test water are neutralized by protons (hydrogen ions) released from the carboxyl group of the amino acid, so that, as the reagent composition is added, the pH of the test water decreases and tends to change toward neutral.

While the amino acid that can be used is not particularly limited, it is preferable to use glycine, proline, or alanine, which are usually inexpensive and readily available.

The pH of the reagent composition added to the test water in step 1 is low because the reagent composition includes the coloring reagents that are acid-dissociated in the test water. However, because coloring reagents are generally unstable under acidic conditions, decomposition may proceed while being preserved or during storage. Thus, preferably, the pH of the reagent composition may be adjusted to be close to neutral by addition of an inorganic strong base, such as sodium hydroxide or potassium hydroxide. Once the pH of the reagent composition has been adjusted to be close to neutral by adding the inorganic strong base, decomposition of the coloring reagents is suppressed, and the reliability of the result of measuring the pH of the test water can be increased.

### EXAMPLE

500 g of the reagent composition with a composition shown in Table 5 was prepared. The reagent composition corresponds to the concrete example of the reagent composition according to the first embodiment.

TABLE 5

| | | | | Mol | |
|---|---|---|---|---|---|
| | | Blending | | concen- | |
| Components | | ratio | | tration | Remarks |
| Solvent | Propylene | Balance | | — | — |
| | glycol | | | | |
| Surfactant | Polyoxyeth- | 0.50 | wt % | — | — |
| | ylene | | | | |
| | octylphenyl | | | | |
| | ether | | | | |
| First | Methyl red | 348 | mg/kg | 1.29 mmol/kg | ×0.348 |
| coloring | | | | | Unit |
| reagent | | | | | concen- |
| | | | | | tration of |
| | | | | | coloring |
| | | | | | reagent |
| Second | Phenol red | 522 | mg/kg | 1.47 mmol/kg | ×0.522 |
| coloring | | | | | Unit |
| reagent | | | | | concen- |
| | | | | | tration of |
| | | | | | coloring |
| | | | | | reagent |
| Third | Bromocresol | 856 | mg/kg | 1.58 mmol/kg | ×0.856 |
| coloring | blue | | | | Unit |
| reagent | | | | | concen- |
| | | | | | tration of |
| | | | | | coloring |
| | | | | | reagent |

Assuming a case in which 100 mL of test water to which 0.75 g of the reagent composition has been added is irradiated with visible light with wavelengths 420 nm, 525 nm, and 590 nm, the absorbance at each wavelength of the visible light that is predicted in this case was calculated in light of the expressions (1), (2), and (3) as well as the Henderson-Hasselbalch's equation. Here, the absorbance at each of the above wavelengths of visible light was calculated with respect to the test water with the pH varying in a range of from 1 to 10 in increments of 0.1.

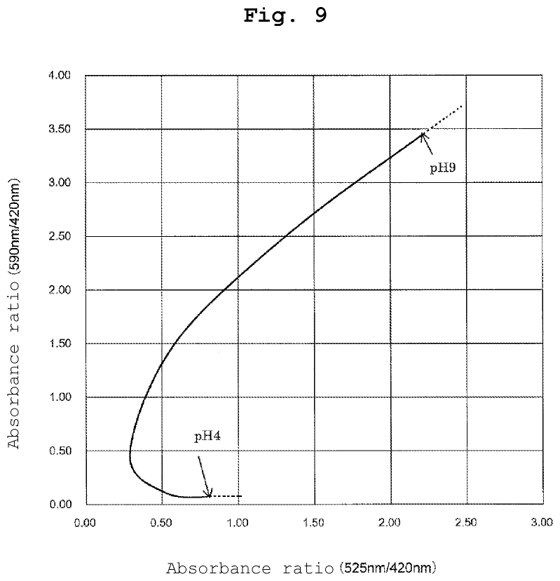

From the calculated absorbance at each wavelength, the relationship between pH values of the test water and an absorbance ratio (525 nm/420 nm), and the relationship between pH values of the test water and an absorbance ratio (590 nm/420 nm) were determined. The results are shown in Table 6-1 to Table 6-4. Further, the relationships between both absorbance ratios and the pH values of the test water were plotted to create a graph for determining the pH of the test water. The results are shown in FIG. 9.

TABLE 6-1

| Calculated absorbance | | | Absorbance ratio | | pH of |
|---|---|---|---|---|---|
| 420 nm | 525 nm | 590 nm | 525 nm/420 nm | 590 nm/420 nm | test water |
| 0.334 | 0.339 | 0.026 | 1.02 | 0.08 | 2.0 |
| 0.333 | 0.334 | 0.026 | 1.00 | 0.08 | 2.1 |
| 0.333 | 0.330 | 0.025 | 0.99 | 0.08 | 2.2 |
| 0.333 | 0.327 | 0.025 | 0.98 | 0.08 | 2.3 |
| 0.332 | 0.324 | 0.025 | 0.97 | 0.08 | 2.4 |
| 0.332 | 0.321 | 0.025 | 0.97 | 0.08 | 2.5 |
| 0.332 | 0.319 | 0.025 | 0.96 | 0.08 | 2.6 |
| 0.332 | 0.318 | 0.025 | 0.96 | 0.08 | 2.7 |
| 0.332 | 0.316 | 0.025 | 0.95 | 0.08 | 2.8 |
| 0.332 | 0.315 | 0.025 | 0.95 | 0.08 | 2.9 |
| 0.332 | 0.314 | 0.025 | 0.95 | 0.08 | 3.0 |
| 0.332 | 0.312 | 0.025 | 0.94 | 0.08 | 3.1 |
| 0.332 | 0.311 | 0.025 | 0.94 | 0.08 | 3.2 |
| 0.332 | 0.310 | 0.025 | 0.93 | 0.08 | 3.3 |
| 0.332 | 0.308 | 0.025 | 0.93 | 0.08 | 3.4 |
| 0.333 | 0.307 | 0.025 | 0.92 | 0.08 | 3.5 |
| 0.333 | 0.305 | 0.026 | 0.92 | 0.08 | 3.6 |
| 0.334 | 0.303 | 0.026 | 0.91 | 0.08 | 3.7 |
| 0.334 | 0.300 | 0.026 | 0.90 | 0.08 | 3.8 |
| 0.335 | 0.296 | 0.026 | 0.88 | 0.08 | 3.9 |
| 0.336 | 0.292 | 0.026 | 0.87 | 0.08 | 4.0 |
| 0.337 | 0.287 | 0.026 | 0.85 | 0.08 | 4.1 |
| 0.339 | 0.281 | 0.026 | 0.83 | 0.08 | 4.2 |
| 0.341 | 0.274 | 0.027 | 0.81 | 0.08 | 4.3 |
| 0.343 | 0.266 | 0.027 | 0.78 | 0.08 | 4.4 |
| 0.345 | 0.256 | 0.028 | 0.74 | 0.08 | 4.5 |
| 0.348 | 0.246 | 0.029 | 0.71 | 0.08 | 4.6 |
| 0.351 | 0.233 | 0.030 | 0.66 | 0.09 | 4.7 |
| 0.354 | 0.220 | 0.032 | 0.62 | 0.09 | 4.8 |

TABLE 6-2

| Calculated absorbance | | | Absorbance ratio | | pH of |
|---|---|---|---|---|---|
| 420 nm | 525 nm | 590 nm | 525 nm/420 nm | 590 nm/420 nm | test water |
| 0.357 | 0.205 | 0.035 | 0.58 | 0.10 | 4.9 |
| 0.360 | 0.190 | 0.038 | 0.53 | 0.11 | 5.0 |
| 0.363 | 0.175 | 0.042 | 0.48 | 0.12 | 5.1 |
| 0.366 | 0.161 | 0.048 | 0.44 | 0.13 | 5.2 |
| 0.368 | 0.147 | 0.055 | 0.40 | 0.15 | 5.3 |
| 0.370 | 0.135 | 0.064 | 0.36 | 0.17 | 5.4 |
| 0.370 | 0.124 | 0.075 | 0.33 | 0.20 | 5.5 |
| 0.369 | 0.115 | 0.089 | 0.31 | 0.24 | 5.6 |
| 0.367 | 0.109 | 0.105 | 0.30 | 0.29 | 5.7 |
| 0.364 | 0.105 | 0.124 | 0.29 | 0.34 | 5.8 |
| 0.360 | 0.103 | 0.145 | 0.28 | 0.40 | 5.9 |
| 0.355 | 0.103 | 0.169 | 0.29 | 0.48 | 6.0 |
| 0.349 | 0.104 | 0.195 | 0.30 | 0.56 | 6.1 |
| 0.342 | 0.108 | 0.223 | 0.32 | 0.65 | 6.2 |
| 0.334 | 0.113 | 0.251 | 0.34 | 0.75 | 6.3 |
| 0.326 | 0.119 | 0.279 | 0.36 | 0.86 | 6.4 |
| 0.318 | 0.125 | 0.307 | 0.39 | 0.97 | 6.5 |
| 0.310 | 0.132 | 0.334 | 0.43 | 1.08 | 6.6 |
| 0.302 | 0.140 | 0.358 | 0.46 | 1.19 | 6.7 |
| 0.294 | 0.147 | 0.381 | 0.50 | 1.30 | 6.8 |
| 0.287 | 0.155 | 0.401 | 0.54 | 1.40 | 6.9 |
| 0.280 | 0.163 | 0.419 | 0.58 | 1.50 | 7.0 |
| 0.272 | 0.172 | 0.435 | 0.63 | 1.60 | 7.1 |
| 0.265 | 0.180 | 0.449 | 0.68 | 1.69 | 7.2 |
| 0.258 | 0.190 | 0.461 | 0.73 | 1.78 | 7.3 |
| 0.251 | 0.200 | 0.471 | 0.80 | 1.88 | 7.4 |
| 0.243 | 0.210 | 0.481 | 0.86 | 1.97 | 7.5 |
| 0.236 | 0.222 | 0.489 | 0.94 | 2.07 | 7.6 |
| 0.228 | 0.233 | 0.496 | 1.02 | 2.18 | 7.7 |

TABLE 6-3

| Calculated absorbance | | | Absorbance ratio | | pH of |
|---|---|---|---|---|---|
| 420 nm | 525 nm | 590 nm | 525 nm/420 nm | 590 nm/420 nm | test water |
| 0.220 | 0.246 | 0.502 | 1.12 | 2.28 | 7.8 |
| 0.212 | 0.258 | 0.508 | 1.22 | 2.40 | 7.9 |
| 0.204 | 0.270 | 0.513 | 1.32 | 2.51 | 8.0 |
| 0.197 | 0.282 | 0.518 | 1.43 | 2.63 | 8.1 |
| 0.190 | 0.293 | 0.522 | 1.54 | 2.75 | 8.2 |
| 0.183 | 0.303 | 0.525 | 1.65 | 2.87 | 8.3 |
| 0.177 | 0.312 | 0.528 | 1.76 | 2.98 | 8.4 |
| 0.172 | 0.320 | 0.531 | 1.86 | 3.08 | 8.5 |
| 0.168 | 0.327 | 0.533 | 1.95 | 3.18 | 8.6 |
| 0.164 | 0.333 | 0.535 | 2.03 | 3.26 | 8.7 |
| 0.161 | 0.339 | 0.537 | 2.11 | 3.34 | 8.8 |
| 0.158 | 0.343 | 0.538 | 2.17 | 3.40 | 8.9 |
| 0.156 | 0.346 | 0.539 | 2.22 | 3.46 | 9.0 |
| 0.154 | 0.349 | 0.540 | 2.27 | 3.50 | 9.1 |
| 0.153 | 0.352 | 0.541 | 2.30 | 3.54 | 9.2 |
| 0.151 | 0.354 | 0.541 | 2.34 | 3.58 | 9.3 |
| 0.150 | 0.355 | 0.542 | 2.36 | 3.60 | 9.4 |
| 0.150 | 0.356 | 0.542 | 2.38 | 3.62 | 9.5 |
| 0.149 | 0.357 | 0.542 | 2.40 | 3.64 | 9.6 |
| 0.148 | 0.358 | 0.543 | 2.41 | 3.65 | 9.7 |
| 0.148 | 0.359 | 0.543 | 2.42 | 3.67 | 9.8 |
| 0.148 | 0.359 | 0.543 | 2.43 | 3.68 | 9.9 |
| 0.148 | 0.360 | 0.543 | 2.44 | 3.68 | 10.0 |
| 0.147 | 0.360 | 0.543 | 2.45 | 3.69 | 10.1 |
| 0.147 | 0.360 | 0.543 | 2.45 | 3.69 | 10.2 |
| 0.147 | 0.361 | 0.543 | 2.45 | 3.70 | 10.3 |
| 0.147 | 0.361 | 0.543 | 2.46 | 3.70 | 10.4 |
| 0.147 | 0.361 | 0.543 | 2.46 | 3.70 | 10.5 |
| 0.147 | 0.361 | 0.544 | 2.46 | 3.70 | 10.6 |

TABLE 6-4

| Calculated absorbance | | | Absorbance ratio | | pH of |
|---|---|---|---|---|---|
| 420 nm | 525 nm | 590 nm | 525 nm/420 nm | 590 nm/420 nm | test water |
| 0.147 | 0.361 | 0.544 | 2.46 | 3.71 | 10.7 |
| 0.147 | 0.361 | 0.544 | 2.46 | 3.71 | 10.8 |
| 0.147 | 0.361 | 0.544 | 2.46 | 3.71 | 10.9 |
| 0.147 | 0.361 | 0.544 | 2.46 | 3.71 | 11.0 |
| 0.147 | 0.361 | 0.544 | 2.47 | 3.71 | 11.1 |
| 0.147 | 0.361 | 0.544 | 2.47 | 3.71 | 11.2 |
| 0.147 | 0.361 | 0.544 | 2.47 | 3.71 | 11.3 |
| 0.147 | 0.361 | 0.544 | 2.47 | 3.71 | 11.4 |
| 0.147 | 0.361 | 0.544 | 2.47 | 3.71 | 11.5 |
| 0.147 | 0.361 | 0.544 | 2.47 | 3.71 | 11.6 |
| 0.147 | 0.361 | 0.544 | 2.47 | 3.71 | 11.7 |
| 0.147 | 0.361 | 0.544 | 2.47 | 3.71 | 11.8 |
| 0.147 | 0.361 | 0.544 | 2.47 | 3.71 | 11.9 |
| 0.147 | 0.361 | 0.544 | 2.47 | 3.71 | 12.0 |
| 0.147 | 0.361 | 0.544 | 2.47 | 3.71 | 12.1 |
| 0.147 | 0.361 | 0.544 | 2.47 | 3.71 | 12.2 |

Waters for verification adjusted to the pH values shown in Table 7 were prepared. The pH of each water for verification was confirmed using a glass electrode manufactured by HORIBA, Ltd (Type: 9625-10D). With respect to 100 mL of each of the waters for verification, 0.75 g of the reagent composition was added and stirred, and then the absorbance of visible light with wavelengths 420 nm, 525 nm, and 590 nm was measured. Then, with respect to each water for verification, an absorbance ratio (525 nm/420 nm) and an absorbance ratio (590 nm/420 nm) were determined, and the pH was determined by applying the absorbance ratios to the graph of FIG. 9. The results are shown in Table 7.

TABLE 7

| | pH (Glass | Absorbance | Absorbance | pH (Value |
|---|---|---|---|---|
| | electrode | ratio | ratio | determined |
| Water for | measurement | (525 nm/ | (590 nm/ | from |
| verification | value) | 420 nm) | 420 nm) | FIG. 9) |
| No. 1 | 10.9 | 2.45 | 3.80 | — |
| No. 2 | 7.8 | 0.82 | 1.85 | 7.3 |
| No. 3 | 7.5 | 0.75 | 1.90 | 7.4 |
| No. 4 | 7.1 | 0.60 | 1.53 | 6.9 |
| No. 5 | 6.6 | 0.35 | 0.90 | 6.3 |
| No. 6 | 6.5 | 0.32 | 0.85 | 6.3 |
| No. 7 | 6.0 | 0.21 | 0.92 | 5.7 |
| No. 8 | 1.8 | 1.15 | 0.06 | — |

1. A method for measuring the pH of test water in a prescribed range, the method comprising:
a step 1 for adding, to the test water, a reagent composition including a first coloring reagent of which absorbance in an ultraviolet-visible region can be varied through acid dissociation in one stage due to a pH variation in the prescribed range, a second coloring reagent of which absorbance in the ultraviolet-visible region can be varied through acid dissociation in one stage due to a pH variation in the prescribed range and which has an acid dissociation constant (pKa) greater than that of the first coloring reagent, and at least one type of a third coloring reagent of which absorbance in the ultraviolet-visible region can be varied through acid dissociation in one stage due to a pH variation in the prescribed range and which has an acid dissociation constant (pKa) between those of the first coloring reagent and the second coloring reagent, each of the first coloring reagent, the second coloring reagent, and the third coloring reagent having an absorbance in the ultraviolet-visible region of greater than zero in the prescribed range; and
a step 2 for measuring, with respect to the test water to which the reagent composition has been added, absorbance at an arbitrary wavelength in the ultraviolet-visible region; and
a step 3 for determining the pH of the test water based on the absorbance measured in step 2.

2. The method for measuring the pH of test water according to claim 1, comprising:
in step 2, measuring the absorbance at each of a plurality of mutually different wavelengths; and
in step 3, determining the pH of the test water in accordance with a correlation analysis result obtained in advance using, as variables, the absorbance at each of the plurality of wavelengths and pH of the test water.

3. The method for measuring the pH of test water according to claim 1, comprising:
in step 2, measuring the absorbance at each of a plurality of mutually different, at least three, wavelengths; and
in step 3, determining the pH of the test water in accordance with a correlation analysis result obtained in advance using, as variables, absorbance ratios in which the denominator is the absorbance with respect to a wavelength among the plurality of wavelengths at which the absorbance is least likely to change due to a variation in the pH of the test water, and in which the numerator each separately is the absorbance with respect to each of the other wavelengths, and pH of the test water.

4. The method for measuring the pH of test water according to claim 3, comprising:
in step 3, provisionally determining, separately for each of the absorbance ratios, a pH of the test water in accordance with a correlation analysis result obtained in advance using, as variables, each one of the absorbance ratios and pH of the test water; and
cancelling step 3 if a difference between the pH of the test water provisionally determined based on one of the absorbance ratios and the pH of the test water provisionally determined based on another one of the absorbance ratios is greater than a prescribed value.

5. The method for measuring the pH of test water according to claim 1, further comprising:
a step 4 in which step 1 to step 3 are repeated at least once to set a function (y=Fx) of a pH (y) of the test water determined in each step 3 and a cumulative added amount (x) of the reagent composition with respect to the test water at the time of the determination as variables, and in which the pH (y) when the added amount (x) is zero in the function (y=Fx) is finally determined as the pH of the test water.

6. The method for measuring the pH of test water according claim 5, comprising also determining a buffer capacity of the test water based on the function (y=Fx).

7. The method for measuring the pH of test water according to claim 6, comprising using, as the reagent composition added to the test water in step 1, one that includes an amino acid if the buffer capacity determined with respect to the test water is greater than a prescribed value.

8. The method for measuring the pH of test water according to claim 2, wherein the prescribed range of pH is from 4 to 9,
the method comprising:
in step 1, using the reagent composition that includes a first coloring reagent selected from those with an acid dissociation constant (pKa) in a range of from 4.1 to 6.0, a second coloring reagent selected from those with an acid dissociation constant (pKa) in a range of from 6.5 to 8.5, and one type of a third coloring reagent selected from those with an acid dissociation constant (pKa) in a range of from 5.5 to 7.5; and
in step 2, measuring absorbance at three wavelengths of a wavelength selected from a range of from 410 to 430 nm, a wavelength selected from a range of from 515 to 535 nm, and a wavelength selected from a range of from 580 to 600 nm.

9. The method for measuring the pH of test water according to claim 2, wherein the prescribed range of pH is from 4 to 12,
the method comprising:
in step 1, using the reagent composition that includes a first coloring reagent selected from those with an acid dissociation constant (pKa) in a range of from 4.1 to 6.0, a second coloring reagent selected from those with an acid dissociation constant (pKa) in a range of from 8.5 to 11.5, and a total of two types of third coloring reagents including a first type of coloring reagent selected from those with an acid dissociation constant (pKa) in a range of from 5.5 to 7.5, and a second type of coloring reagent selected from those with an acid dissociation constant (pKa) in a range of from 7.0 to 9.5 and having an acid dissociation constant (pKa) greater than that of the first type of coloring reagent; and
in step 2, measuring absorbance at four wavelengths of a wavelength selected from a range of from 410 to 430 nm, a wavelength selected from a range of from 450 to 470 nm, a wavelength selected from a range of from 515 to 535 nm, and a wavelength selected from a range of from 580 to 600 nm.

10. The method for measuring the pH of test water according to claim 3, further comprising:
a step 4 in which step 1 to step 3 are repeated at least once to set a function (y=Fx) of a pH (y) of the test water determined in each step 3 and a cumulative added amount (x) of the reagent composition with respect to the test water at the time of the determination as variables, and in which the pH (y) when the added amount (x) is zero in the function (y=Fx) is finally determined as the pH of the test water.
